(19) United States
(12) Patent Application Publication
(10) Pub. No.: US 2021/0220510 A1
(43) Pub. Date: Jul. 22, 2021

(54) Cellularised Dressing and Method for Producing Same

(71) Applicants: Urgo Recherche Innovation et Developpement, Chenove (FR); Poietis, Pessac (FR)

(72) Inventors: Delphine Fayol, Merignac (FR); Fabien Guillemot, Preignac (FR); Catherine van der Mee, Bordeaux (FR); Christelle Laurensou, Dijon (FR)

(73) Assignees: Urgo Recherche Innovation et Developpement, Chenove (FR); Poietis, Pessac (FR)

(21) Appl. No.: 16/972,105

(22) PCT Filed: Jun. 6, 2019
(86) PCT No.: PCT/FR2019/051367

§ 371 Date: Dec. 4, 2020

(30) Foreign Application Priority Data
Jun. 7, 2018 (FR) 18 54944

(51) Int. Cl. A61L 15/42; A61L 15/40; A61L 15/44; A61L 15/26; A61F 13/00

(52) U.S. Cl. CPC A61L 15/425; A61L 15/40; A61F 13/00987; A61L 15/26; A61L 15/44

(57) ABSTRACT

The present invention deals with a cellularized dressing and the method for production of such a dressing, where this method preferably comprises a step of bioprinting of cells.

The present invention deals with a cellularized dressing and the method for production of such a dressing, where this method preferably comprises a step of bioprinting of cells.

Cellularized dressings or skin substitutes are known and have been sold for a long time. The advantage of cellularized dressings resides in the fact that the exogenous supply of living cells participates in the wound healing. The cells supplied by the dressing directly or indirectly participate (via the secretion of factors) in the healing process.

Such dressings generally come in the form of at least one resorbable material (i.e. at least one material normally present in the cellular environment). The Grafix® products sold by Osiris, can be indicated as examples. These products are composed of a placental membrane comprising an extracellular matrix (ECM) rich in collagen, growth factors, fibroblasts, mesenchymal stem cells and epithelial cells. The Apligraf® product sold by Organogenesis and composed of keratinocytes, fibroblasts and bovine collagen can also be mentioned. As for the Dermagraft® product sold by Advanced Biohealing, it contains dermal derivatives and human fibroblasts.

Such cellularized dressings, although having a proven effectiveness for healing, may however present the risks of virus transmission such as, for example, prions (in particular for cellularized dressings comprising compounds of animal origin).

Also, in a cellularized dressing, the cellular density, the zone of localization of the cells or the homogeneous distribution of cells are poorly controlled parameters.

There is therefore a need for new cellularized dressings not having the disadvantages from the prior art.

The printing of cells, also called bioprinting, directly on bioresorbable materials in order to reconstitute a dermis or an epidermis is known and described in the patent application WO2016/115034 from Wake Forest University or in the patent application WO20160/073782 from Organovo. These patent applications cover the printing of cells and extracellular components (such as for example collagen, hyaluronic acid, etc.) for producing skin ex vivo. The materials or substrates onto which the cells are printed are materials conventionally used in cell cultures (e.g. hydrogels containing collagen, hyaluronane, polyethylene glycol, etc.). The patent application WO2016/115034 also describes a biomask comprising a hydrogel layer containing cells inside of this hydrogel, where said hydrogel is next bioprinted onto a polyurethane structure. This patent application therefore does not describe the bioprinting of cells onto the polyurethane structure. In fact, there is no direct contact between the bioprinted cells and a non-resorbable material. Additionally, in the patent application WO2016/115034 the polyurethane structure corresponds to a bioprinted polyurethane gel; this polyurethane structure therefore does not allow absorption of exudates.

Biomaterials and tissue engineering are also known for replacing a portion or a function of an organ or a tissue.

Biomaterials are synthetic or living materials that can be used for medical purposes for replacing a portion or a function of an organ or tissue. Said biomaterials must meet several obligations:
be well tolerated by the receiver, meaning not causing infection, inflammation, allergy or even rejection response if it involves a living material;
not contain toxic substances, such as endocrine disruptors or cancerogenic agents;
address mechanical constraints for adapting to pressures exerted by the environment (blood pressure for vascular prostheses, millions of openings and closings for a heart valve, weight of the body for hip or knee prosthetics, etc.);
shapable, implantable or injectable, degradable (resorbable) or not according to the case, possibly porous so that they can be colonized once implanted, etc.

The tissue engineering itself consists of production of a tissue by multiplication of cells around a matrix or a scaffolding. The concrete implementation however runs up against various problems. For example, in an artificial environment the cells tend to lose their ability to differentiate. Further the cells sometimes express atypical proteins which, after implantation, can lead to inflammation or to rejection reactions.

The use of non-resorbable materials comprising cells for therapeutic purposes is therefore described in the prior art (for example in the case of production of a tissue with the help of a scaffold matrix). However, when this matrix is not resorbable, it is intended to remain in place within the organism for at least a long time and it is not intended to be removed. It is the same for the use of non-resorbable biomaterials.

In contrast, the present invention relates to the use of non-resorbable materials (preferably synthetic materials) for therapeutic purposes, but said materials are not intended to replace a portion or a function of an organ or tissue. They are not intended to remain in place within the organism; they are intended to be withdrawn after regeneration of the organ or tissue onto which they were implanted. According to the invention, the materials thus have a role as temporary dressing.

The bioprinting of cells such as described in the applications WO2016/115034 and WO2016/073782 is done on bioresorbable materials. Methods for bioprinting are also described in the applications WO2011/107599, WO2016/097619 and WO2016/097620. These applications in particular describe that bioprinting may be used to produce tissues (for example implantable tissues for regenerative medicine).

Bioprinting of cells on non-resorbable materials intended to be used temporarily is therefore not described in the prior art.

In the context of the present invention, cells are thus printed on non-resorbable materials. Said materials are used as dressings. Such materials are not naturally present in the cellular environment and are not typically used in cellular culture. Surprisingly, the inventors observed that cells bioprinted on such materials were not only viable, capable of proliferating, but were also capable of migration. The advantage of such a printing or bioprinting is that it is possible to personalize or adapt the dressing to each patient and each wound, thus allowing made-to-measure treatment in order to optimize the healing of wounds. Thus, according to the healing phase that the wound is in, it is possible to integrate dermal cells and epidermal cells or only one of these two cell types. Within a single dressing, it is also possible to vary the cellular density from one area to another in order to optimize the treatment according to the morphology of the wound. Additionally, cellularized dressings according to the present invention serve to avoid the risks of virus transmission, in particular because they do not contain compounds of animal origin. The bioprinting also serves to precisely locate a zone on the dressing on which the cells will be present at a controlled concentration. For dressings which have quadrille grid of fibers, the cells can be specifically printed on the grid, or off the grid. The precision of this technique is of order of tens of microns. The cellular density, the zone of localization of the cells and/or the homogeneous distribution of cells are thus better controlled in the dressings according to the invention as compared to cellularized dressings from the prior art.

In a first aspect, the invention thus relates to a cellularized dressing intended to be temporarily applied to a wound, where said dressing comprises cells on a non-resorbable material.

For the purposes of the present invention, “cellularized dressing” is understood to mean that the dressing comprises cells.

According to the invention, the expression “intended to be temporarily applied to a wound” means that the dressings are intended to be removed from the wound. This expression also means that the dressings according to the invention have a shape suited for temporary application to a wound. The dressings according to the invention in fact have a protective role and are intended to be withdrawn once the organ or tissue of the wound is regenerated. The dressings according to the invention are not resorbed and are not intended to be kept in place for a long time (several days or several weeks). Advantageously the dressing covers all or part of the wound, preferably all of the wound.

According to the invention, the expression “said dressing comprises cells on a non-resorbable material” means that the cells are in direct contact with the non-resorbable material. Advantageously, the cells are therefore not mixed with a hydrogel or incorporated inside a hydrogel. According to an embodiment of the invention, said dressing is therefore free of hydrogels.

For the purposes of the present invention, “non-resorbable material” means that the material is not progressively eliminated within the wound, unlike resorbable materials which themselves breakdown naturally. The removal/breakdown of a non-resorbable material therefore requires a physical/mechanical action, unlike the breakdown of a resorbable material.

The non-resorbable material advantageously has the following properties: 1) it allows absorption of exudates; and/or 2) it can undergo a dimensional change (by gelling or deformation related to the absorption); and/or 3) it does not adhere to the tissues; and/or 4) it is preferably partially hydrophilic in the hydrated state; and/or 5) it has a slickness in the hydrated state; and/or 6) it is not cytotoxic. According to the invention, “slickness in the hydrated state” means that the material has a surface condition which does not allow cells to adhere thereto but which still keeps them alive.

Advantageously, said non-resorbable material is selected from:
an interface dressing;
an absorbent dressing; or
a hydrophilic polyurethane foam.

Advantageously, said non-resorbable material is a material comprising fibers, in particular the interface dressing or the absorbent dressing.

Advantageously, the non-resorbable material allows absorption of exudates. Preferably, the non-resorbable material according to the invention is a hydrophilic polyurethane foam allowing the absorption of exudates.

For example, an interface dressing is such as described in the patent application EP 2,793,773; meaning an adhering interface dressing comprising: i) a non-adhering cohesive gel formed from a hydrophobic elastomeric matrix made up of a styrene-(ethylene-butylene)-styrene or styrene-(ethylene-propylene)-styrene triblock elastomer, which could be combined with a styrene-(ethylene-butylene) or styrene-(ethylene-propylene) diblock copolymer, where said elastomer is highly plasticized by means of a mineral oil, and containing as a dispersion a small amount of hydrophilic particles of a hydrocolloid; and ii) a flexible open-mesh fabric, said fabric comprising yarns which are coated with the non-adhering cohesive gel so as to leave the meshes essentially unobstructed, characterized in that the fabric is a heat-set knit with weft yarns, said yarns being continuous yams with nonelastic filaments, which have an extensibility in the transverse direction, measured according to standard EN 13726-4, of between 0.01 and 0.5 N/cm. According to a preferred embodiment, said non-adhering cohesive gel is formed from a hydrophobic elastomeric matrix comprising for 100 parts by weight of elastomer selected from a styrene-(ethylene/butylene)-styrene or styrene-(ethylene/propylene)-styrene triblock elastomer which could be associated with a styrene-(ethylene/butylene) or styrene(ethylene/propylene) diblock copolymer, 1000 to 2000 parts by weight of a paraffin oil, and containing in dispersion from 2 to 20% by weight, relative to the total weight of the elastomer matrix, of hydrophilic particles of a hydrocolloid.

As an example, an absorbent dressing is such as described in the patent application EP 2,696,828, meaning an adhesive absorbent dressing comprising an absorbent nonwoven 6) and a protective support that is impermeable to fluids and permeable to water vapor 4), characterized in that: i) the support is formed by assembling a continuous film 4a) and an openwork reinforcement that is coated, on at least one of the surfaces thereof, with adhesive silicone gel 4b) without blocking the openings in the reinforcement, said reinforcement covering the entire surface of the film, ii) in that said dressing further comprises a non-absorbent web 5) and a complementary nonwoven 7) which are secured to each other along their periphery while encasing said absorbent nonwoven, preferably without point of attachment therewith, and iii) in that said non-absorbent web 5) adheres to the adhesive silicone gel 4b) coated on said reinforcement.

According to an embodiment of the invention, the cells present within the dressing are cells adhering to a substrate (for example polystyrene in a culture box or flask). They are in particular chosen from cells from the dermis or epidermis. They are in particular chosen from fibroblast type cells and/or epithelial type cells. Advantageously the cells are chosen from fibroblasts and/or keratinocytes, in particular the primary fibroblasts and/or the primary keratinocytes. Even more advantageously, the cells are chosen from primary dermal fibroblasts and/or primary epidermal keratinocytes. The term “fibroblasts” refers to cells with bare spindle shape, irregularly shaped, which are responsible for the formation of fibers. In cell cultures, many other cell types cannot be morphologically distinguished from fibroblasts. In organ and tissue cultures in which the relations between cells are retained, it is possible to identify the fibroblasts by means of accepted histological criteria. The term “epithelial cells” refers to cells opposite each other which form a continuous tissue similar to a mosaic with very little intercellular substances as can be seen in in vitro cultures of tissues and organs. The term “fibroblast type cells” refers to cells attached to a substrate and which appear elongated and bipolar. In cell cultures, various cell types have similar morphologies. The cells which take irregular shapes or spindle shapes are often qualified as fibroblasts. The term “epithelial type cells” refers to cells which are attached to a substrate and which appear flat and polygonal. In cell cultures, epithelial cells can take various shapes but tend to form a tissue of packed polygonal cells.

According to an embodiment of the invention, in said dressing, the cells are (or previously were) bioprinted on said non-resorbable material. In this embodiment, the cells are also directly bioprinted on said non-resorbable material, and said dressing therefore does not comprise a bioprinting hydrogel. Some dressings have the property of not adhering to wounds, and the cells do not adhere to the materials generally used in dressings. It is therefore complicated to make cells live on the surface of this type of dressing because the cells will not be able to adhere to it. One of the advantages of bioprinting is that it serves to print cells on the surface of this type of dressing, and to keep them there until transfer from the dressing to the wound. As examples, the applications WO2016/115034, WO20160/073782 WO2011/107599, WO2016/097619 and WO2016/097620 describe methods for bioprinting which can be used for bioprinting a dressing according to the invention.

According to an embodiment of the invention, in said dressing, the cells are present (or were bioprinted) near fibers of said material (i.e. on the fibers themselves) or inside one or more motifs defined in the fibers. Advantageously, according to the invention, the cells can thus be present (or be bioprinted) near fibers with a concentric motif, a radial motif, a geometric motif, or a random nongeometric motif (meaning not representing a geometric shape), or inside at least one of these motifs. According to an even more preferred embodiment, in said dressing, the cells are present (or were bioprinted) near fibers of said material, at the intersection of the fibers of said material and/or at the center of each grid square of said material.

According to an embodiment of the invention, said dressing is saturated with liquid up to 90% of the absorption capacity thereof. Preferably, said dressing is saturated with liquid at a level included between at least 50% of the absorption capacity thereof, preferably at least 80% and up to 90% of the absorption capacity thereof. According to the invention “between at least 50% and up to 90%” is understood to mean all the values included between 50% and 90% and in particular 50%, 55%, 60%, 65%, 70%, 75%, 80%, 85% and 90%. The dressing according to the invention must also perform the functions of absorption or gelling of the exudates. During the addition of the cells on the non-resorbable material or during the bioprinting of cells, only a few picoliters of cellular ink are deposited or printed. The cells must be in an environment saturated with moisture or even a liquid environment in order to be able to survive and grow. It must therefore be possible to maintain cellular viability, while also allowing the dressing to provide these functions. It is therefore important to find a balance between the absorption or gelling of the exudates by the dressing and cellular survival. Preferably, the dressing will therefore need to be sufficiently hydrated (but not to saturation) so that the cells on the surface thereof survive, and thus promote healing. The inventors have observed that the dressing according to the present invention specifically met this balance when the dressing is saturated with liquid to 90% of the absorption capacity thereof The absorption capacity of the dressing is measured according to the standard NF EN 13726-1.

According to an embodiment of the invention, said dressing comprises a cellular concentration included between 50 and 30,000 cells/cm², preferably between 200 and 20,000 cells/cm².

According to an embodiment of the invention, said dressing further comprises an active ingredient, preferably an active ingredient having a favorable role in the treatment of wounds. Advantageously, said active ingredient is chosen from an antiseptic, an antibacterial, an antibiotic, a pain reliever, an anti-inflammatory, an anesthetic or a compound which promotes healing of the wound. As examples, the antibacterials/antibiotics may be derivatives of silver such as silver salts or of other metals (for example silver sulfate, chloride or nitrate, and silver sulfadiazine), complexes of silver or other metals (for example silver zeolites such as AlphaSan or ceramics), metronidazole, neomycin, Polymyxin B, penicillins (amoxicillin), clavulanic acid, tetracyclines, minocycline, chlorotetracycline, aminoglycosides, amikacin, gentamicin or probiotics. The antiseptics can be chlorhexidine, triclosan, biguanides, hexamidine, thymol, lugol, povidone iodine, benzalkonium chloride and benzethonium. The pain relievers can be acetaminophen, codeine, dextropropoxyphene, tramadol, morphine and derivatives thereof, and corticosteroids and derivatives. The anti-inflammatories can be glucocorticoids, non-steroidal anti-inflammatories, aspirin, ibuprofen ketoprofen, flurbiprofen, diclofenac, aceclofenac, ketorolac, meloxicam, piroxicam, tenoxicam, naproxen, indomethacin, naproxcinod, nimesulide, celecoxib, etoricoxib, parecoxib, rofecoxib, valdecoxib, phenylbutazone, niflumic acid mefenamic acid. Other active ingredients promoting healing can also be used, for example retinol, vitamin A, vitamin D N-acetyl-hydroxyproline, Centella asiatica extracts, papain, essential oils of thyme, of niaouli, of rosemary and of sage, hyaluronic acid, polysulfated oligosaccharides and salts thereof (in particular synthetic sulfated oligosaccharides having 1 to 4 oses units such as the potassium salt of octasulfated sucrose or the silver salt of octasulfated sucrose), sucralfate, allantoin, urea, metformin, enzymes (for example proteolytic enzymes such as streptokinase, trypsin or collagenase), peptides or protease inhibitors. Anesthetics such as benzocaine, lidocaine, dibucaine, pramoxine hydrochloride, bupivacaine, mepivacaine, prilocaine, or etidocaine can also be used.

According to an embodiment, the invention also relates to a kit comprising a) a dressing according to the invention and b) an active ingredient such as indicated above.

The dressing according to the invention may also comprise any other material conventionally used by the person skilled in the field of dressings, for example at least one protective pouch or culture box or any system with which to make handling thereof or transfer thereof easier.

In a second aspect, the invention also relates to the method for production of a dressing such as defined above. Example 1 illustrates a method to produce a dressing according to the invention.

In an embodiment, the invention thus relates to a method for production of a cellularized dressing such as defined above, comprising a method of bringing cells into contact, advantageously into direct contact, with a non-resorbable material. This step of bringing into contact may consist of a direct application of cells with the non-resorbable material, a step of impregnation or a step of printing.

According to a preferred embodiment, the step of bringing into contact is a step of bioprinting of cells on said non-resorbable material. In particular, the method for production covers the printing of two cellular types (primary fibroblasts and primary keratinocytes) on the three materials: an interface dressing, an absorbent dressing, a hydrophilic polyurethane (HPU) foam. The step of bioprinting is done with a bio-ink comprising cells to be printed.

Advantageously, the method for production of a dressing according to the invention does not comprise a step of bioprinting of a hydrogel. Even more advantageously, the method for production of a dressing according to the invention does not comprise a step of bioprinting a hydrogel which was mixed with cells or a hydrogel in which cells were incorporated.

According to a preferred embodiment, the bioprinting of cells is done with a bio-ink in which the cells are in suspension or in form of aggregates. Advantageously, said bio-ink consists of a culture medium comprising a concentration of suspended cells between 0.1×10⁶ to 100×10⁶, preferably 1×10⁶ to 80×10⁶. The bio-ink may be prepared according to the foregoing protocol: after a cellular culture step (for example under conventional conditions known to the person skilled in the art), the cells intended to be bioprinted are gathered up, and then centrifuged (for example at 400 G for five minutes). The cellular plug is next recovered, then suspended in a culture medium with a cellular density of 70×10⁶ cells/mL. The bio-ink can also have the form of aggregates (or micro-aggregates) of cells. In this embodiment, the cell concentration is greater than 100×10⁶ cells/mL. These aggregates may have, for example, the form of those described in the patent application WO2016/089825.

Advantageously, during the step of bioprinting, said non-resorbable material is moist or dry, preferably moist. Even more advantageously, said material is moist or dry when the bioprinting step is done on the interface dressing. Alternatively, said material is moist when the bioprinting step is done on the absorbent dressing or on the hydrophilic polyurethane foam.

Before the bioprinting step, the dressings can be prepared, in particular, cut as needed under sterile conditions.

Possibly, before the bioprinting step, said dressings can be moistened by using a culture medium (for example with 1 to 2 mL of culture medium for a dressing of about 1.5 cm×1.5 cm), and then, as needed, the excess culture medium can be absorbed. Advantageously, when the interface dressing is moistened, it is preferable to absorb the excess culture medium before the bioprinting step.

According to a preferred embodiment, the method according to the invention comprises the following steps:
optionally, a step of cellular culture of cells intended to be bioprinted;
optionally, a step of preparation of a bio-ink comprising cells intended to be bioprinted;
optionally, a step of moistening of non-resorbable material by means of a culture medium;
a step of bioprinting of cells on said non-resorbable material.

According to a preferred embodiment, said non-resorbable material is bioprinted near the fibers of said material or inside one or more motifs defined by the fibers. More advantageously, according to the invention, the motif may typically be a concentric motif, a radial motif, a geometric motif, or a random nongeometric motif (meaning not representing a geometric shape) or inside at least one of these motifs.

According to a preferred embodiment, said non-resorbable material is bioprinted near fibers of said material, at the intersection of the fibers of said material and/or at the center of each grid square of said material.

In a third aspect, the invention also relates to the use of a dressing such as defined above.

The invention thus relates to a method for treating a patient's wound comprising:
the topical administration, on the wound intended to be treated, of the cellularized dressing such as defined above;
optionally, the removal of said dressing.

According to an embodiment, said method may also comprise the following steps:
the topical administration, on the wound intended to be treated, of a first cellularized dressing such as defined above;
the removal of the first dressing;
the topical administration, on the wound intended to be treated, of a second cellularized dressing such as defined above;
optionally, the removal of the second dressing.

In a fourth aspect, the invention also relates to a kit intended to obtain a cellular dressing according to the present invention, where said kit comprises:

a) a non-resorbable material; and

b) cells intended to be placed in contact with (or bioprinted on) the non-resorbable material, where said cells are in a medium appropriate to cellular survival.

According to the invention, “medium appropriate to cellular survival” means for example an appropriate culture medium. Such media are known to the person skilled in the art.

In this aspect, the dressing according to the invention can more specifically be adapted to the wound of the patient to be treated and prepared just before administration thereof.

Said kit intended to get a cellularized dressing according to the present invention may also comprise an active ingredient such as mentioned above, or also any other material conventionally used by the person skilled in the field of dressings, for example at least one protective pouch or culture box or any system with which to make handling thereof or transfer thereof easier.

The invention will be better illustrated by the following examples and figures. The following examples aim to clarify the subject matter of the invention and illustrate advantageous embodiments. These figures do not aim to restrict the scope of the invention.

### FIGURES

FIG. 1 shows the first printing motif on the Urgotul® interface dressing: the printing spots are positioned at the intersection of fibers.

FIG. 2 shows the second printing motif on the Urgotul® interface dressing: printing spots are added on each of the intersection of fibers.

FIG. 3 shows the printing spots of the motif on the absorbent dressing: the printing spots are positioned at the intersection of fibers, on the fibers themselves, and at the center of each grid square.

FIG. 4 shows the results of printing and seeding controls of primary fibroblasts on the interface, the absorbent dressing and the HPU foam, when the dressings are moist. ***means that p<0.001.

FIG. 5 shows the results of printing and seeding controls of primary fibroblasts on the interface, the absorbent dressing and the HPU foam, when the dressings are dry. ***means that p<0.001.

FIG. 6 represents the normalized results from FIG. 4 where a corresponds to the results obtained with the Urgotul® interface dressing, b the Urgotul Absorb® absorbent dressing and c the HPU foam. ***means that p<0.001.

FIG. 7 represents the normalized results from FIG. 5 where a corresponds to the results obtained with the Urgotul® interface dressing, b the Urgotul Absorb® absorbent dressing and c the HPU foam. ***means that p<0.001.

FIG. 8 shows the results of printing and seeding controls of primary keratinocytes on the moist Urgotul® interface and on the moist HPU foam. *means that p<0.05.

FIG. 9 shows the normalized results for the viability of keratinocytes in which a shows the results obtained with the Urgotul® interface and b with the HPU foam. *means that p<0.05.

FIG. 10 shows the immunolabeling results for collagen I from the fibroblasts printed on the moistened interface (c, d) and the HPU foam (e, f) and the control fibroblasts at the bottom of the culture well (a, b).

FIG. 11 shows the immunolabeling results for the fibronectin synthesized by the fibroblasts on the moistened interface (c, d) and the HPU foam (e, f) and by the control fibroblasts at the bottom of the culture well (a, b).

FIG. 12 shows the immunolabeling results for collagen III synthesized by the fibroblasts printed on the moistened interface (c, d) and the HPU foam (e, f) and by the control fibroblasts at the bottom of the culture well (a, b).

FIG. 13 shows the immunolabeling results for Ki67 antigen present in the nucleus of the proliferating fibroblasts printed on the moistened interface (c, d) and the HPU foam (e, f) and by the proliferating control fibroblasts (a, b).

FIG. 14 shows the percentage of Ki67 labeled cells.

FIG. 15 shows the results of printing and seeding controls of primary keratinocytes on the interface and on the HPU foam. *means that p<0.05.

FIG. 16 shows the number of days that the keratinocytes needed to migrate from the dressings (interface and the HPU foam) on which they were printed or controls.

FIG. 17 shows the immunolabeling results for the Ki67 antigen present in the nucleus of the proliferating keratinocytes printed on the moistened interface (c, d) and the HPU foam (e, f) and by the proliferating control keratinocytes (a, b).

FIG. 18 shows the 100% confluent keratinocyte lawn only below the dressing, obtained after 8 days of migration from the samples of the HPU foam.

### EXAMPLES

### Example 1: Method for Production of a Dressing According to the Present Invention

The two cellular types used are primary dermal fibroblasts and primary epidermal keratinocytes extracted from operatory collections (mammary and foreskin plastic surgeries).

1. Cellular Culture

The DMEM culture medium for fibroblasts is composed of 10% fetal calf serum and 1% antibiotics: penicillin, streptomycin, amphotericin.

The culture medium for keratinocytes is the CNT-PR medium sold by CellnTec.

The culture media for these two cell types are changed every 2 to 3 days.

2. Preparation of the Bio-Ink

Before use, the fibroblasts and keratinocytes are detached from the culture flask with trypsin/0.25% EDTA and fetal calf serum is added after separation of the cells for stopping the enzymatic reaction. Counting with trypan blue is done for counting the population and determining the cellular viability. The cells are then centrifuged at 400 G for 5 minutes. The printing ink is prepared by suspending the cellular plug in the culture medium at the density of 70×10⁶ cells/mL.

3. Fluorescence Labeling of the Cells

The cells could be labeled with a fluorescent cellular tracer, CellTracker™ orange CMRA Dye (ThermoFischer Scientific, catalog number C34551) for viewing the cells after printing. In this case, the cellular plug produced after trypsinization is suspended in the CMRA cellular tracer and the cells are placed in the incubator at 37° C. for 15 minutes and then centrifuged again.

4. Preparation of the Dressings

The three dressings (Urgotul® interface dressing, the Urgotul Absorb® absorbent dressing and the HPU foam) are cut under sterile conditions using a scalpel (about 1.5 cm×1.5 cm) and positioned in the wells of 12 well culture plates. In the case where the dressings are moistened, 1 mL of culture medium is deposited on the Urgotul® interface and 2 mL is deposited on the Urgotul Absorb® absorbent dressing and on the HPU foam which are thicker. At the end of 20 minutes, the culture medium is withdrawn from each of the culture wells containing the dressings in order to be able to position the culture plate during the printing step. In the case of the Urgotul® interface, it is preferable (and in some cases necessary) to put the dressing down on a sterile compress before printing so that the excess culture medium between the grid of fibers can be absorbed. In fact, if the medium is still present within the grid of the interface, the imaging system has difficulty detecting the dressing.

5. Cellular Seeding

The bioprinting of cells done in this example uses the laser aided bioprinting mode of the printer such as described in the patent applications WO2011/107599, WO2016/097619 and WO2016/097620. This bioprinting method requires the prior creation of a printing file containing the set of instructions to be executed by the machine. The motif (geometry and spacing of points) is part of the information contained in this file. The bio-ink is first placed on a cartridge made up of a glass slide covered with a very thin layer of gold. During printing, the laser beam passes through this cartridge and reaches the area of the bio-ink. A cavity forms and spreads for finally generating a jet which causes the formation of a drop of liquid and depositing of the drop on the receiver. By moving over the donor slide, the laser beam generates drops which are deposited on the receiver according to a predefined cellular motif. This laser-assisted bioprinting method relies on physical phenomena of laser-matter interaction and involves many parameters. Some are set during the design of the machine (like, for example, the wavelength of the laser), and others can be adjusted by the operator according to the printing conditions (like, for example, the energy of the laser).

In this example, the adjustable parameters from Table 1 below were held at fixed value. The motif was thus the only variable parameter during the bioprinting. This motif was created from the image of the receiving substrate and customized according to the geometric properties of the support. It was thus possible to print specifically on the grid of the interface and the absorbent dressing.

TABLE 1
The printing parameters set during the bioprinting step done in the examples

| | | Cellular | Distance | Thickness | Donor/ | Volume of ink |
|---|---|---|---|---|---|---|
| | Pulse | concentration | to the | of the | receiver | deposited on |
| Energy | length | in the bio-ink | focal plane | bio-ink layer | distance | the donor slide |
| 30 μJ | 200 ns | 70 million | −100 μm | 45 μm | 500 μm | Between 8 |
| | (T8) | cells/mL | | | | and 8.5 μL |

Imaging System and a Software Tool

An imaging system and a software tool were developed for automatically creating personalized printing motifs based on the observable grid on the dressing materials. With this tool the printing zones can be lined up with the observable grids on the dressings (Urgotul® interface dressing and Urgotul Absorb® absorbent dressing). It is also possible to vary the cellular density by fiber by modulating the spacing of the printing spots.

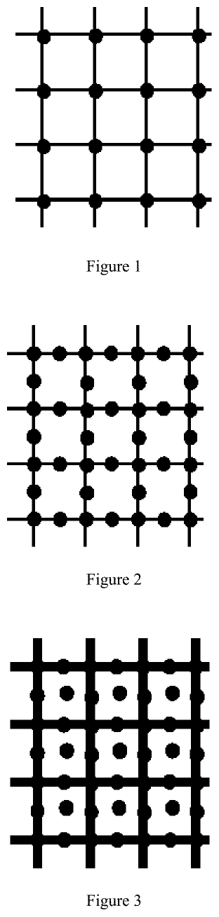

Two motifs were chosen for the Urgotul® interface. In the first, the printing spots are positioned at the intersection of the fibers (FIG. 1). The second motif is created by adding spots on each of the fibers (FIG. 2). For the absorbent dressing, the spots of the motif are located at the intersection of fibers, on the fibers themselves, and at the center of each grid square (FIG. 3).

The dressing is placed at the bottom of a well of a culture plate (12 well plate). Then, the imaging system acquires and reconstructs an image so as to return the entirety of the surface of the material (12 photos in total). During the second step, the software renders the image obtained in the first step in black and white by detecting the dark zones and the light zones. After this rendering, the hollows (=centers of the grids) appear in black and the solids (=fibers) in white. The coordinates of the centers of the dark zones are then calculated. These points are located on a fiber. By iterating analogously with the calculated points, it is possible to create motifs with a variable point density by fiber. The motif produced by the algorithm can then be loaded into the printer software in order to use it as a template for printing of the cells.

The imaging software must satisfy two objectives in order to be confirmed.

In a first step, the calculation must lead to a good positioning of the points on the dressing, in order to reproduce the desired motif. This objective was met during development of the software. The imaging of the interface dressing is used to generate images with a higher contrast than with the absorbent dressing. Insufficient contrast is a source of errors in the calculation of the positioning of the points, which is the case with the absorbent dressing. An intermediate solution was found: a motif correction function was added to the software. It serves to manually remove and add points and therefore to correct errors in the calculation of the motif case-by-case.

In a second step, the proper positioning of the bioprinted drops on the dressing support must be validated. In order to be sure of correctly visualizing the result of the printing, primary fibroblasts and primary keratinocytes labeled with orange florescent tracer were printed in place of the hydrogel initially planned. The motifs of keratinocytes printed on the interface using the software specifically serve to position the cellular spots on the fibers of the interface. Whatever the cellular type printed, the software leaves the choice of the motif to the user. For example, the cellular spots can be positioned automatically at the intersection of each fiber of the dressing, or else the user can themselves position the cellular response to the predefined distance (300-500-800 μm, etc.). In this example, the motive printed on the HPU foam is a 1 cm² square with a 200 μm spacing between the cellular spots (keratinocytes or fibroblasts).

### Example 2: Test of Cellular Viability

Before printing, the dressings are either dry or moistened. During seeding of the controls, 30,000 cells in 7.5 μL of culture medium are deposited on each of the dressings. Immediately after printing of the cells, or depositing them by pipette (for the controls), the dressings are submerged in 2 mL of culture medium and are “flushed” (in order to recover the maximum the cells on the materials, successive “flushes” are done using a pipette). The cells are next labeled and counted on a Malassez chamber. The labeling is done directly on the cells after printing. The cells are left in culture (after printing) for a minimum of 24 hours before doing the cellular viability test. The keratinocytes or fibroblasts in solution are then seeded in a new culture well and are placed in an incubator at 37° C. and 5% CO₂. After 24 hours, the culture medium is withdrawn and the cells are labeled with calcein and ethidium solution. The cellular viability percentage is calculated after having counted the number of living cells and dead cells in six zones per culture well.

In a first step, the “live dead” technique is done on the printed or control primary fibroblasts on the dry and moist Urgotul® interface, Urgotul Absorb® absorbent dressing or HPU foam. The “live dead” technique serves to distinguish living cells from dead cells within a single culture. The ubiquitous intercellular esterase activity and the presence of an intact plasma membrane are characteristics of living cells. These cells transform the acetoxymethyl calcein (AM) nonfluorescent coloring into fluorescent calcein (green). The dead cells are characterized by a loss of integrity of their plasma membrane. The ethidium homodimer-1 (EthD-1) enters the cells and bonds with the nucleic acids which consequently have a red fluorescence.

In a second step, the cellular viability was studied with the printed or control primary keratinocytes on the moist Urgotul® interface and HPU foam.

The statistical test used for analyzing the counting results of the cellular viability is a Student test was a value is 0.05.

A. Result of the Viability of Fibroblasts

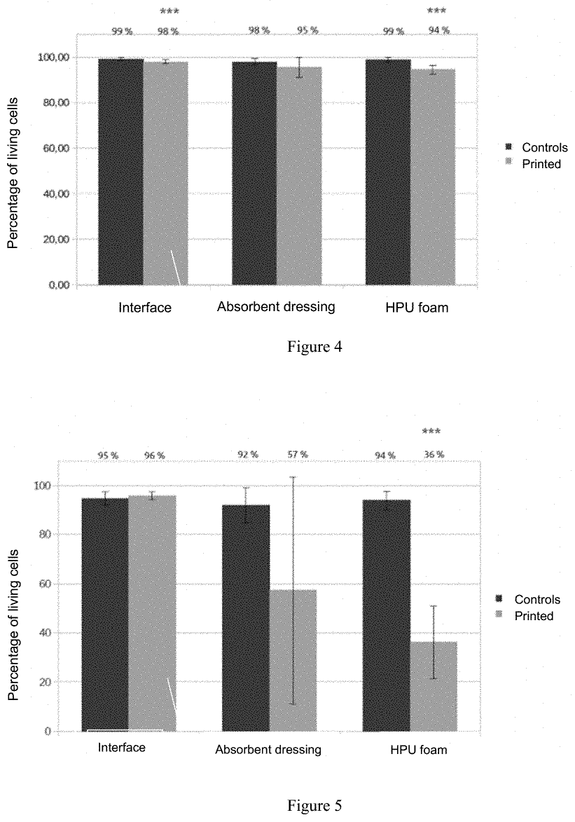

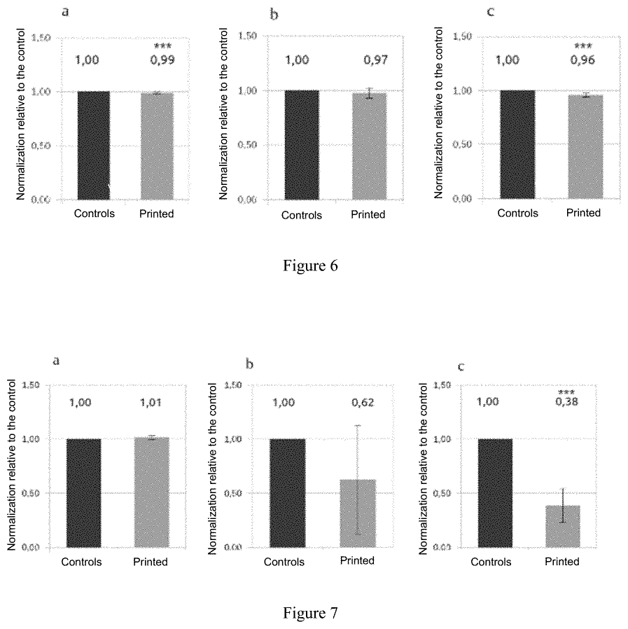

The results of printing and of seeding controls of primary fibroblasts on the interface, the absorbent dressing and the HPU foam are shown in FIG. 4 (with moist dressings) and FIG. 5 (with dry dressings). The normalized results are shown in FIGS. 6 and 7 where a corresponds to the results obtained with the Urgotul® interface dressing, b the Urgotul Absorb® absorbent dressing and c the HPU foam.

When the fibroblasts are printed on the moistened interface, absorbent dressing and HPU foam, the viability of the fibroblasts is over 94%, and also very close to that of the control cells. The printed and control fibroblasts on these three moist dressings remain viable. The small value of the standard deviations proves that these results are reproducible.

In contrast, the viability results for printing on the dry dressings are very variable aside from the Urgotul® interface. The viability of printed or control cells on the dry interface is close to the results on the moist interface. The cells therefore remain viable after printing on the moist or dry Urgotul® interface. The control cells on the dry Urgotul Absorb® absorbent dressing and the dry HPU foam give viability results comparable to the results on the same dressings when moist. The viability results for fibroblasts printed on the Urgotul Absorb® absorbent dressing are highly variable. The result is 57%±46%. Finally, the cells printed on the dry HPU foam have a 36% cellular viability. A little more than half of the cells die after being printed on this dry dressing compared to the same dressing when moist. Generally, the cells do not support dry environments and printing media well, which may explain this cellular viability difference.

B. Result of the Viability of Keratinocytes

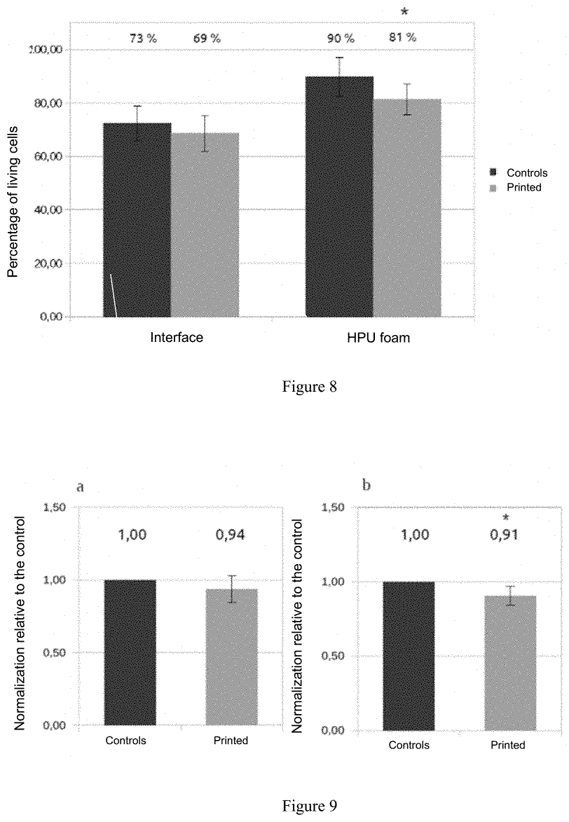

The results of printing and seeding controls of primary keratinocytes on the moist Urgotul® interface and on the moist HPU foam are shown in FIG. 8. FIG. 9 shows the normalized results for the viability of keratinocytes in which a shows the results obtained with the Urgotul® interface and b with the HPU foam.

The cells printed on the moist Urgotul® interface have a viability close to that of the control cells on the interface, with about 70%±7% viability. Since between the control cells and the printed cells the viabilities are very close, printing on this support is not the cause of the 30% dead cells.

The percentage viability from the printing of primary keratinocytes on moist HPU foam is 81%±6%. The control cells on this same dressing have a 90%±7% viability. The difference between these two values is significant.

The normalization of the results shows that the viability of keratinocytes printed on the Urgotul® interface and on the HPU foam are very close to the viability of the controls.

### Example 3: Test of Cellular Migration

Before printing the dressings are either dry or moistened. The control cells are seeded on the dressings with 30,000 cells in 7.5 μL of culture medium. After the step of printing cells and depositing cells by pipette (controls), the dressings are stored either 30 minutes or 3 hours in an incubator at 37° C. and 5% CO₂. This period is called the storage time. Each dressing is next turned over (printed surface against the culture well) and submerged in 2 mL of culture medium. A stainless steel ring is placed on each dressing so that it doesn't float. The culture medium is changed every 2 to 3 days. The dressings are kept in culture for 4 days for the primary fibroblasts and 8 days for the primary keratinocytes (migration time necessary to arrive at 50% confluence), in order to be able to subsequently label and immunolabel the cells which migrated from the dressings onto the plastic surface of the culture wells. The conditions tested with the moist dressings are the same as those with the dry dressings.

A. Results of the Migration of Fibroblasts

No migration of printed fibroblasts from the moist Urgotul® interface was observed even though control fibroblasts migrated at the end of only one day.

It took 4 and 9 days for the printed fibroblasts to migrate from the moist Urgotul Absorb® absorbent dressings after a wait for 30 minutes or 3 hours after printing. The control cells on this moist dressing took a comparable time for migrating: 4 days and 5 days with a wait respectively for 30 minutes and 3 hours after printing.

It only takes printed and control fibroblasts one day for migrating from a moist HPU foam with storage for 30 minutes. Finally, when this storage time extends to 3 hours, the control fibroblasts take 5 days for migrating and no migration is observed from the dressings on which the fibroblasts were printed.

The control fibroblasts overall take more time to migrate from the Urgotul® interface, the Urgotul Absorb® absorbent dressing and the HPU foam if the storage time is 3 hours. This result seems similar to the Urgotul Absorb® absorbent dressing and the HPU foam when the fibroblasts were printed. They take almost twice as long to migrate from the absorbent dressing and they do not migrate from the HPU foam. The 30-minute storage time therefore seems better suited to cells printed on the moist dressings.

No migration was observed from the dry Urgotul® interface, the Urgotul Absorb® absorbent dressing and the HPU foam when the storage time is 3 hours even though the printed and control fibroblasts migrate from all the dry dressings when the storage time is 30 minutes. This storage time can be increased (beyond 30 minutes) by using a larger seeding volume (7.5 μL<V<1 mL), while remaining below the hydration volume at saturation so that the cellularized dressing can still play its role of absorbing exudates from the wound.

The times for migration of bioprinted fibroblasts from the moist Urgotul® interface and the HPU foam were studied after 30-minutes storage time. Additional results were acquired in that way for 30 samples per condition. For each sample, it is observed over a period of 4 days whether the bioprinted cells migrate outside of the dressing.

Under these conditions, for nearly all HPU foam samples on which fibroblasts were printed, the migration was observed starting at 2 days.

The results for migration of fibroblasts from the Urgotul® interface are more variable. The migration is observed at the end of 2 days after printing for many of the interface samples (19 out of 30 samples). At the end of 4 days, the fibroblasts started to migrate from 4 samples, and no migration was observed from 7 samples.

B. Results of the Migration of Keratinocytes

For 50% of the Urgotul® interface dressings on which the keratinocytes were printed and deposited by pipette, no migration was observed. From 50% of the remaining samples, the migration of keratinocytes was observed between 2 and 4 days after printing or manual seeding of the cells. The migration time from the Urgotul® interface is relatively short but this migration is only seen in half of the Urgotul® interface samples.

From 19 samples of HPU foam on which keratinocytes were printed or controls, migration was observed between 2 and 4 days. The migration of printed keratinocytes was not observed on one HPU foam sample which is negligible. The migration time for the printed and control keratinocytes from the HPU foam is short and involves nearly all of the samples.

The keratinocytes printed on the Urgotul® interface and the HPU foam just like the control keratinocytes for the most part express the Ki67 antigen.

After 8 days of migration from HPU foam samples, surprisingly a 100% confluent keratinocyte lawn was observed (for 2 wells of 4) only under the dressing. On the surface of this lawn, some keratinocytes were starting to proliferate and stratify. In that case, the keratinocytes proliferated a lot. Because of contact inhibition a portion of these cells did not express the Ki67 antigens and returned to quiescent phase. Other cells continued to proliferate and to divide at the surface of the confluent lawn.

The proliferation percentage (cells which express the Ki67 antigen) is calculated in order to be able to quantify the expression of the Ki67 antigen and compare the printed cells with the control cells. The control keratinocytes have a 68%±18% proliferation percentage. This large variability can be explained by a seeding density of keratinocytes that was too low (2000 cells/cm²).

The proliferation percentage of keratinocytes printed on the interface is 92%.

The proliferation percentage of keratinocytes printed on the HPU foam is 80%±18%. This result is comparable to the proliferation percentage of control keratinocytes. The keratinocytes printed on the HPU foam did not experience any change in their capacity to proliferate.

### Example 4: Labeling and Immunolabeling of Cells

After printing and depositing by pipette of fibroblasts or keratinocytes on the moistened Urgotul® interface and the HPU foam with 30-minutes storage time, the dressings were turned over (printed side against the bottom of the culture well) for 4 days for the fibroblasts and 8 days for the keratinocytes. The cells are next fixed, and then, in order to verify that cellular metabolism is not affected by contact with the dressing after printing, immunolabeling of the cells is done.

The labelings done are:
Actin, collagen I, collagen III, fibronectin and Ki67 on the fibroblasts; and
Ki67 on the keratinocytes.

Observation of the actin filaments is done by labeling with phalloidin. The phalloidin coupled with a red fluorescent stain (Texas red) binds to the actin filaments and prevents their depolarization. The actin filaments then appear fluorescent in red.

The cells are fixed with 4% formaldehyde. The cellular membranes are made permeable with the use of the Triton solution, and then a treatment with BSA (bovine serum albumin) serves to reduce the nonspecific attachments. The cells are next labeled with phalloidin and then observed under a fluorescence microscope.

A. Immunolabeling of Collagen I, Collagen III and Fibronectin

Collagen I and III are fibrillary polypeptides synthesized and secreted by the primary fibroblasts of the dermis. Their role is to participate in the elasticity and strength of the extracellular matrix of the dermis.

Fibronectin is a glycoprotein also synthesized and secreted by the primary fibroblasts of the dermis. It participates in cellular adhesion and migration in the extracellular matrix.

The three labeled proteins are located in the cellular cytoplasm. If no labeling is observed that is because the cells are not expressing and not synthesizing the targeted proteins.

The cells are fixed and the cellular membranes made permeable with methanol. The nonspecific attachment sites are saturated with BSA solution and then the cells are labeled in a first step with the primary antibodies, and then in a second step with the secondary antibodies (which fixes on the first antibody for fluorescing) and DAPI (which labels the cellular nuclei blue). The cells are then observed under fluorescence microscope.

B. Immunolabeling of the Ki67 Antigen

Ki67 is the antigen of a nuclear protein present in proliferating cells in phase G1, S, G2 and M. Cells in quiescence phase G0 do not express this nuclear protein. This labeling is located in the cellular nuclei. If some cells do not express this antigen, it is because the cells are not proliferating. In order to quantify the results, the percentage by number of cells in proliferating phase is calculated.

C. Results

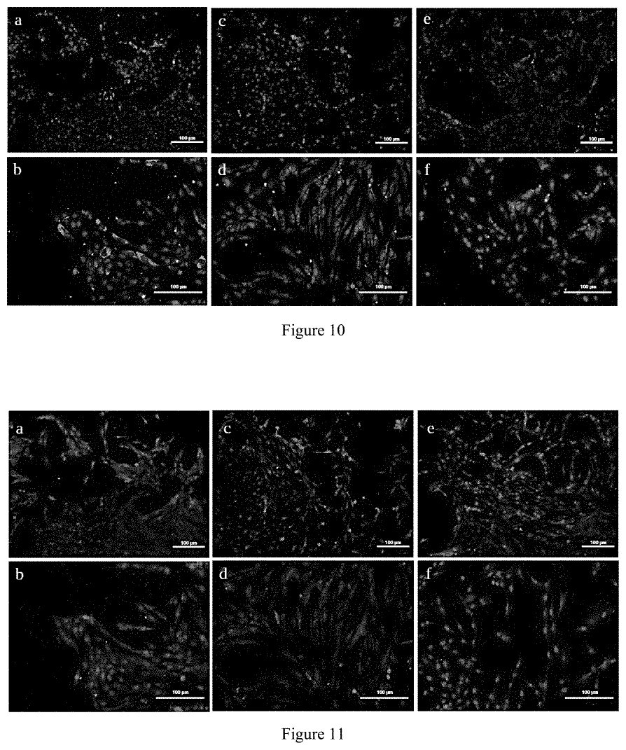

FIG. 10 shows the immunolabeling results for the collagen I from the fibroblasts printed on the moistened interface (c, d) and the HPU foam (e, f) and the control fibroblasts at the bottom of the culture well (a, b). The cells printed on the interface and the HPU foam and also the control cells express collagen I. The intensity of the labeling is stronger in the cytoplasm of some cells, which could be explained by the greater synthesis of collagen I. This intensity difference is observed in the printed fibroblast population on both dressing types (interface and HPU foam) and controls.

FIG. 11 shows the immunolabeling results for the fibronectin synthesized by the fibroblasts on the moistened interface (c, d) and the HPU foam (e, f) and by the control fibroblasts at the bottom of the culture well (a, b). No immunolabeling difference targeting the synthesis of this protein was observed between the printed fibroblasts and the control fibroblasts. Therefore, printing on the interface and on the HPU foam does not disturb the synthesis of fibronectin by the fibroblasts.

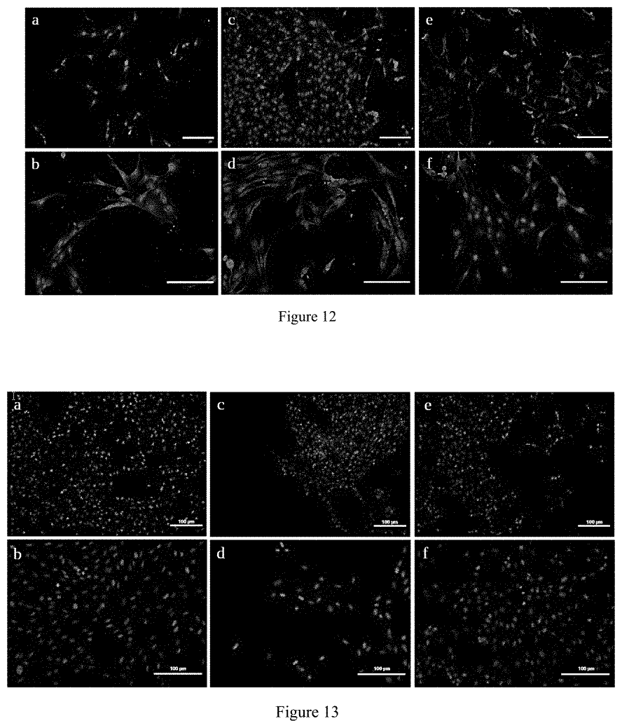

FIG. 12 shows the immunolabeling results for collagen III synthesized by the fibroblasts printed on the moistened interface (c, d) and the HPU foam (e, f) and by the control fibroblasts at the bottom of the culture well (a, b). Just as with the preceding results for immunolabeling of collagen I and fibronectin present and synthesized in the cytoplasm of the fibroblasts, collagen III is also correctly present in the fibroblasts printed on the interface and the HPU fall and in the controls. The immunolabeling results for fibroblasts printed on the two interface and HPU foam dressings and also the control fibroblasts are similar. Printing on these two materials therefore does not disturb the synthesis by the fibroblasts of these proteins which have a central role in the formation of the extracellular matrix in the dermis. The Ki67 antigen is only present in the nuclei of proliferating cells. Labeling them makes it possible to compare the levels of proliferating cells between the fibroblasts printed on the interface and the HPU foam with the control fibroblasts.

FIG. 13 shows the immunolabeling results for the Ki67 antigen present in the nucleus of the proliferating fibroblasts printed on the moistened interface (c, d) and the HPU foam (e, f) and by the proliferating control fibroblasts (a, b). Whatever the condition tested, cells in quiescence phase (unlabeled nuclei) are observed. In some cases, a contact inhibition could explain this non-proliferating state of the cells. Quantitatively, the printed fibroblasts, just like the control fibroblasts, express the Ki67 antigen and are therefore for the most part in proliferation phase.

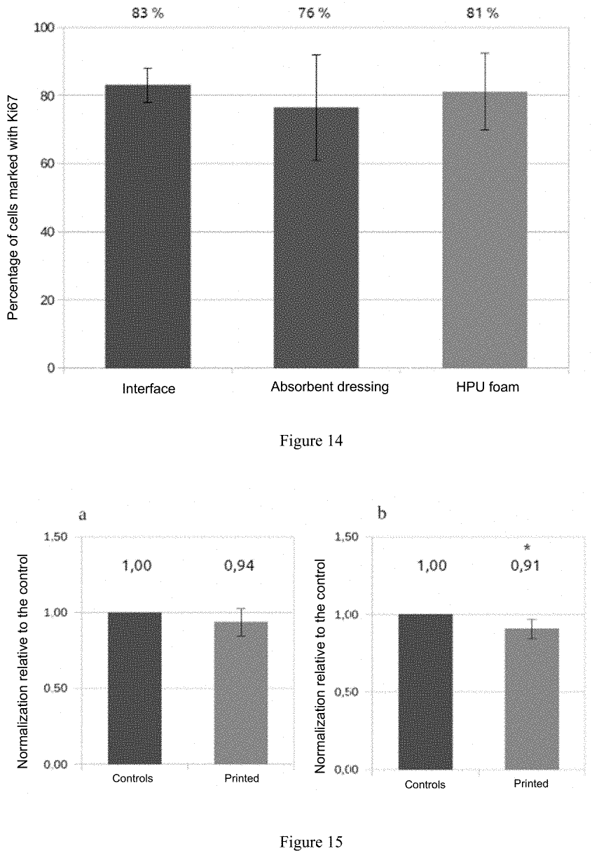

The percentage of labeled cells is calculated in FIG. 14 in order to be able to quantify the expression of the Ki67 antigen and compare the printed cells with the control cells. For the most part, the control fibroblasts are proliferating with 83% of the cells counted expressing the Ki67 antigen. The results for the printed cells fluctuate between 65% to 90% expressing the Ki67 antigen depending on the samples. The average level of proliferating cells among the printed cells having migrated from the interface (76%±15%) or from the HPU foam (81%±11%) is comparable to that of the control cells (83%±5%). In fact, the standard deviations in the percentages of expression of the Ki67 antigen by the printed fibroblasts on the two dressings are relatively large, which brings the results for the printed cells closer to the results for the control cells.

No qualitative or quantitative difference was therefore seen between the cells printed on the interface and the fibroblasts printed on the HPU foam. The proliferating metabolism of the fibroblasts printed on these two moist dressing materials works correctly. The normalized viability of the fibroblasts is very high (over 95%) when the cells are printed on the moist dressings. When the dressings are dry, the viability remains high for the interface but drops a lot for the absorbent and HPU foam dressing.

The results of the tests of migration from the moist dressings with a 30-minute storage time are the most persuasive. These parameters seem to be the best suited to the survival and migration of the fibroblasts from the dressings on which they were printed.

The labeling and immunolabeling give similar results between the printed cells and the control cells. The printing of fibroblasts on the moist interface and HPU foam does not change the synthesis of actin, collagen I and III, fibronectin and Ki67 antigen by the fibroblasts. The metabolism of primary fibroblasts printed on the moist interface and HPU foam is therefore not changed and remains comparable to the metabolism of primary fibroblasts not printed and which grow on the surface of a culture well.

All printing of primary keratinocytes is done on the moistened interface and HPU foam with a 30-minute storage time after printing in an incubator at 37° C. with 5% CO₂.

The results of printing and seeding controls of primary keratinocytes on the interface and on the HPU foam are shown in FIG. 15. The cells printed on the interface have a viability close to that of the control cells on the interface, with about 70%±7% viability. Since between the control cells and the printed cells the viabilities are very close, printing on this support is not the cause of the 30% dead cells. The percentage viability from printing of primary keratinocytes on HPU foam is 81%±6%. The control cells on this same dressing have a 90%±7% viability. The difference between these two values is significant. The normalization of the results shows that the viability of keratinocytes printed on the interface and on the HPU foam are very close to the viability of the controls.

FIG. 16 shows the number of days that the keratinocytes needed to migrate from the dressings (interface and the HPU foam) on which they were printed or controls. For 50% of interface dressings on which the keratinocytes were printed and deposited by pipette, no migration was observed. From 50% of the remaining samples, the migration of keratinocytes was observed between 2 and 4 days after printing or manual seeding of the cells. The migration time from the interface is relatively short but this migration is only seen in too few interface samples. From 19 samples of HPU form on which keratinocytes were printed or controls, migration was observed between 2 and 4 days. The migration of printed keratinocytes was only observed from one HPU foam sample which is negligible. The migration time for the printed and control keratinocytes from the HPU foam is short and involves nearly all of the samples.

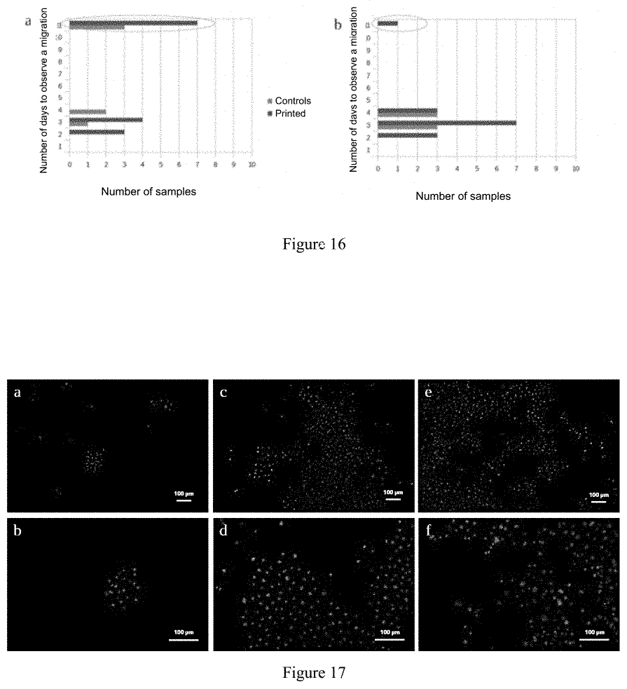

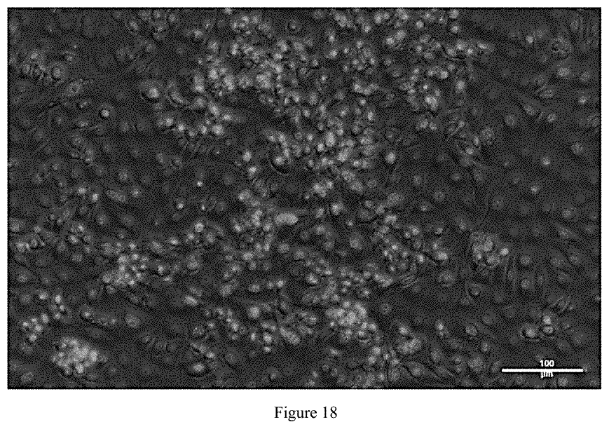

FIG. 17 shows the immunolabeling results for the Ki67 antigen present in the nucleus of the proliferating keratinocytes printed on the moistened interface (c, d) and the HPU foam (e, f) and by the proliferating control keratinocytes (a, b). According to the observations from FIG. 17, the keratinocytes printed on the interface and the HPU foam just like the control keratinocytes for the most part express the Ki67 antigen. Some cells whose nucleus is blue do not express the Ki647 antigen and are observed among the printed keratinocytes but also among the control keratinocytes.

After 8 days of migration from HPU foam samples, surprisingly a 100% confluent keratinocyte lawn was observed (for 2 wells of 4) only under the dressing (FIG. 18). On the surface of this lawn, some keratinocytes were starting to proliferate and stratify. In that case, the keratinocytes proliferated a lot. Because of contact inhibition a portion of these cells did not express the Ki67 antigens and returned to quiescent phase. Other cells continued to proliferate and to divide at the surface of the confluent lawn.

The proliferation percentage (cells which express the Ki67 antigen) is calculated in FIG. 18 in order to be able to quantify the expression of the Ki67 antigen and compare the printed cells with the control cells. The control keratinocytes have a 68%±18% proliferation percentage. This large variability can be explained by a seeding density of keratinocytes that was too low (2000 cells/cm²). The proliferation percentage of keratinocytes printed on the interface is 92%. The proliferation percentage of keratinocytes printed on the HPU foam is 80%±18%. This result is comparable to the proliferation percentage of control keratinocytes. The keratinocytes printed on the HPU foam did not experience any change in their capacity to proliferate. The viability of the keratinocytes printed on the interface is close to the viability of the control keratinocytes. After the study of the migration of keratinocytes (printed or control) from the interface, it was observed that the keratinocytes only migrate from the interface one out of two times. In the case where the keratinocytes have migrated from the interface, the cells grew very well during the 8 to 10 days migration time and started to cover over the surface of the culture well. The results of the proliferation percentage calculations following immunolabeling of the Ki67 antigen indicate that the proliferation of viable keratinocytes is very good. For the most part, the keratinocytes are in proliferation phase 8 days after printing on the interface. Seeding by printing or by deposit with pipette on the interface seems to affect the primary keratinocytes because their viability is less than that on the HPU foam. In contrast, cells migrating from the dressing have a high proliferation level. Two hypotheses can explain this phenomenon:
The first hypothesis is that the primary keratinocytes is a fragile cell type. The stress induced on this material by seeding or printing may therefore cause significant mortality, the time that the cells adapt to this material.
The primary keratinocytes definitely have difficulties adhering to this type of support. The non-proliferating, differentiated keratinocytes which have more difficulty adhering may not survive printing or manual seeding on this dressing. The second hypothesis is therefore that the interface selected the keratinocytes whose metabolism is the most effective with the greatest capacity for proliferation.

With an 81% viability percentage, in large part the keratinocytes survive printing on the HPU foam. This result is comparable to the viability percentage of control keratinocytes on the same material. Printing just like depositing by pipette on this support therefore does not disrupt the survival of the primary keratinocytes. The keratinocytes migrate from the HPU foam at the end of 2 to 4 days after the step of printing or depositing by pipette. The cells are therefore not affected by the culture over several days in this dressing. They migrate quickly and colonize the entire surface of the culture well covered by the HPU foam. The proliferation of the printed keratinocytes takes place correctly and seems to increase on contact with the HPU foam.

1. A cellularized dressing intended to be temporarily applied to a wound, wherein said dressing comprises cells on a non-resorbable material.

2. The cellularized dressing according to claim 1, wherein said non-resorbable material is selected from:
an interface dressing;
an absorbent dressing; or
a hydrophilic polyurethane foam.

3. The cellularized dressing according to claim 1, wherein the cells are chosen from fibroblast type cells and/or epithelial type cells.

4. The cellularized dressing according to claim 1, wherein the cells are chosen from fibroblasts and/or keratinocytes, in particular the primary fibroblasts and/or the primary keratinocytes.

5. The cellularized dressing according to claim 1, wherein the cells are bioprinted on said non-resorbable material.

6. The cellularized dressing according to claim 1, wherein the cells are present near fibers of said material or inside one or more motifs defined by said fibers.

7. The cellularized dressing according to claim 6, wherein the cells are present near fibers with a concentric motif, a radial motif, a geometric motif, or a random nongeometric motif, or inside at least one of these motifs.

8. The cellularized dressing according to claim 6, wherein the cells are present near fibers of said material, at the intersection of the fibers of said material and/or at the center of each grid square of said material.

9. The cellularized dressing according to claim 1, wherein said dressing is saturated with liquid up to 90% of the absorption capacity thereof.

10. The cellularized dressing according to claim 1,
wherein said dressing comprises a cellular concentration included between 50 and 30,000 cells/cm².

11. The cellularized dressing according to claim 1, wherein said dressing further comprises an active ingredient.

12. A method for production of a cellularized dressing according to claim 1, comprising a step of bringing cells into contact with a non-resorbable material.

13. The method for production of a cellularized dressing according to claim 12, wherein the step of bringing cells into contact with a non-resorbable material is a step of bioprinting of cells is done with a bio-ink in which the cells are in suspension or in form of aggregates.

14. The method for production of a cellularized dressing according to claim 12, wherein the non-resorbable material is moist or dry.

15. A kit intended to get a cellularized dressing according to claim 1, said kit comprising:
a) a non-resorbable material; and
b) cells intended to be placed in contact with the non-resorbable material, where said cells are in a medium appropriate to cellular survival.

16. The cellularized dressing according to claim 4, wherein the cells primary fibroblasts and/or primary keratinocytes.

17. The cellularized dressing according to claim 10, wherein said dressing comprises a cellular concentration included between 200 and 20,000 cells/cm².

18. The cellularized dressing according to claim 11, wherein said active ingredient is an antiseptic, an antibacterial, an antibiotic, a pain reliever, an anti-inflammatory, an anesthetic or a compound which promotes healing of the wound.

19. A method for production of a cellularized dressing according to claim 12, wherein the step of bringing into contact is a step of bioprinting of cells on said non-resorbable material.
